(19) United States
(12) Patent Application Publication
(10) Pub. No.: US 2023/0149573 A1
(43) Pub. Date: May 18, 2023

(54) APPARATUS FOR PROVIDING ANTIBACTERIAL FILM

(71) Applicants: Jeong Won HAN, Seoul (KR); Hye Won HAN, Seoul (KR)

(72) Inventors: Jeong Won HAN, Seoul (KR); Hye Won HAN, Seoul (KR)

(21) Appl. No.: 17/855,499

(22) Filed: Jun. 30, 2022

(30) Foreign Application Priority Data
Nov. 12, 2021 (KR) 10-2021-0155581

(51) Int. Cl. A61L 2/10

(52) U.S. Cl. CPC A61L 2/10; A61L 2202/11; B66B 1/466

(57) ABSTRACT

An apparatus for providing an antibacterial film according to an embodiment of the present disclosure makes it possible to place an antibacterial film over a button on a manipulation panel through an antibacterial film providing unit and an antibacterial film recovery unit, thereby preventing the spread of bacteria that may be transmitted by many users' button manipulation. Additionally, it is possible to automatically replace the antibacterial film that covers the button on the manipulation panel at a predetermined time interval set through an auto-timer unit, thereby improving the convenience of use. Additionally, it is possible to sterilize the antibacterial film after use through a sterilization unit provided in the antibacterial film recovery unit for reuse, thereby achieving resource savings.

### CROSS-REFERENCE TO RELATED APPLICATION AND CLAIM OF PRIORITY

This application claims the benefit under 35 USC § 119 of Korean Patent Application No. 10-2021-0155581, filed on Nov. 12, 2021, in the Korean Intellectual Property Office, the entire disclosure of which is incorporated herein by reference for all purposes.

### BACKGROUND

### 1. Field

The present disclosure relates to an apparatus for providing an antibacterial film, and more particularly, to an apparatus for providing an antibacterial film to a manipulation panel having at least one button used to select a target.

### 2. Description of the Related Art

In general, a manipulation unit of an elevator has buttons, and the buttons are push buttons that are controlled to turn on/off by mechanical forces applied to the buttons.

Additionally, the elevator buttons may have raised and recessed braille for visually impaired people.

Accordingly, visually impaired people can identify a desired button through braille code, and manipulate the elevator by pressing the corresponding button.

Since common buttons or braille buttons for visually impaired people are operated by users' physical contact, the elevator buttons are colonized by many bacteria due to the contact with many users' body parts or belongings.

To solve this problem, elevator buttons may be cleaned or sterilized through an ultraviolet sterilizer, but this method lasts only for a short time, and thus it requires effort for periodic management and additional tasks, which incurs economic and temporal costs spending on human inputs.

Additionally, users cannot know whether the elevator buttons are sterilized or disinfected or have been sterilized or disinfected, causing anxiety and inconvenience to hygiene each time the users use the elevator buttons.

### SUMMARY

The present disclosure is designed in the above-described circumstance, and therefore the present disclosure is directed to providing an apparatus for providing an antibacterial film in which the antibacterial film is placed over a button on a manipulation panel through an antibacterial film providing unit and an antibacterial film recovery unit, in order to prevent the spread of bacteria that may be transmitted by many users' button manipulation.

The present disclosure is further directed to providing an apparatus for providing an antibacterial film in which the antibacterial film that covers the button on the manipulation panel is automatically replaced at a predetermined time interval set through an auto-timer unit, in order to improve the convenience of use.

The present disclosure is further directed to providing an apparatus for providing an antibacterial film in which the antibacterial film after use is sterilized through a sterilization unit provided in the antibacterial film recovery unit for reuse, in order to achieve resource savings.

The object of the present disclosure is not limited thereto, and other objects not mentioned herein will be clearly understood by those skilled in the art from the following description.

To achieve the object, an embodiment of the present disclosure provides an apparatus for providing an antibacterial film to a manipulation panel having at least one button used to select a target, including an antibacterial film providing unit including a first winding unit on which an antibacterial film before use is wound, to provide an antibacterial film in use such that the antibacterial film fed from the first winding unit covers the button on the manipulation panel; and an antibacterial film recovery unit including a second winding unit on which an antibacterial film after use is wound.

Additionally, there is provided the apparatus for providing an antibacterial film in which the antibacterial film providing unit further includes a push unit to press down an upper surface of the antibacterial film at an area of the antibacterial film fed from the first winding unit to bring the antibacterial film fed from the first winding unit into close contact with the button on the manipulation panel.

Additionally, there is provided the apparatus for providing an antibacterial film in which the antibacterial film recovery unit further includes an auto-timer unit to operate the second winding unit to wind the antibacterial film after use at a set predetermined time interval.

Additionally, there is provided the apparatus for providing an antibacterial film in which the antibacterial film recovery unit further includes a third winding unit to wind the antibacterial film after use fed from the second winding unit; and a sterilization unit to sterilize the antibacterial film after use wound on the third winding unit.

Additionally, there is provided the apparatus for providing an antibacterial film in which the sterilization unit includes an ultraviolet light source to emit ultraviolet radiation to the antibacterial film after use.

According to an embodiment of the present disclosure, it is possible to place the antibacterial film over the button on the manipulation panel through the antibacterial film providing unit and the antibacterial film recovery unit, thereby preventing the spread of bacteria that may be transmitted by many users' button manipulation.

Additionally, it is possible to automatically replace the antibacterial film that covers the button on the manipulation panel at the predetermined time interval set through the auto-timer unit, thereby improving the convenience of use.

Additionally, it is possible to sterilize the antibacterial film after use through the sterilization unit provided in the antibacterial film recovery unit for reuse, thereby achieving resource savings.

### BRIEF DESCRIPTION OF THE DRAWINGS

FIG. 1 is a front view showing an example of an apparatus for providing an antibacterial film according to an embodiment of the present disclosure installed on a manipulation panel.

FIG. 2 is a side view of FIG. 1.

FIG. 3 is a view showing an example of operation of a push unit in FIG. 2.

FIG. 4 is a diagram showing a sterilization unit of an antibacterial film recovery unit.

FIG. 5 is a block diagram of an apparatus for providing an antibacterial film according to an embodiment of the present disclosure.

### DETAILED DESCRIPTION

Hereinafter, some embodiments of the present disclosure will be described in detail through the exemplary drawings. It should be noted that in adding the reference signs to the elements of each drawing, like reference signs denote like elements as possible even though they are indicated on different drawings. Additionally, in describing the present disclosure, when a certain detailed description of relevant known elements or functions is determined to obscure the subject matter of the present disclosure, the detailed description is omitted.

Furthermore, in describing the elements of the present disclosure, the terms ‘first’, ‘second’, A, B, (a), (b), and the like may be used. These terms are only used to distinguish one element from another, and the nature of the corresponding element or its sequence or order is not limited by the term. It should be understood that when an element is referred to as being “connected”, “coupled” or “linked” to another element, it can be directly connected or linked to other element, but intervening elements may be “connected”, “coupled” or “linked” between each element.

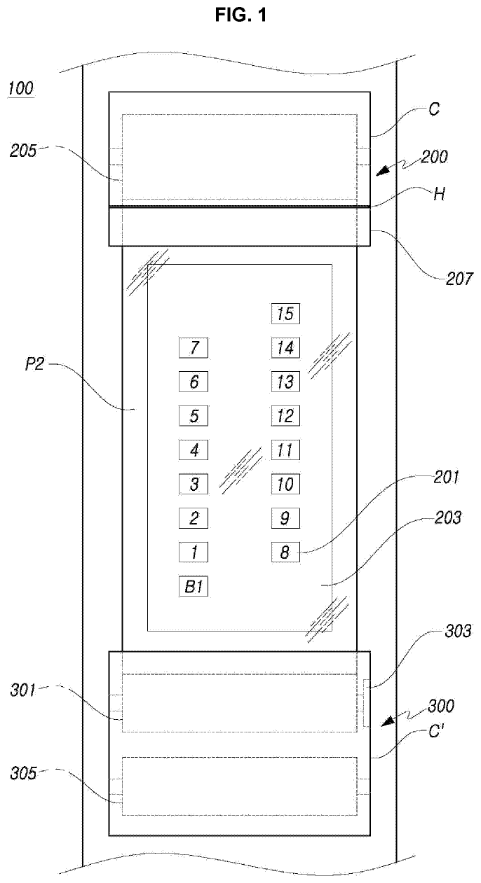

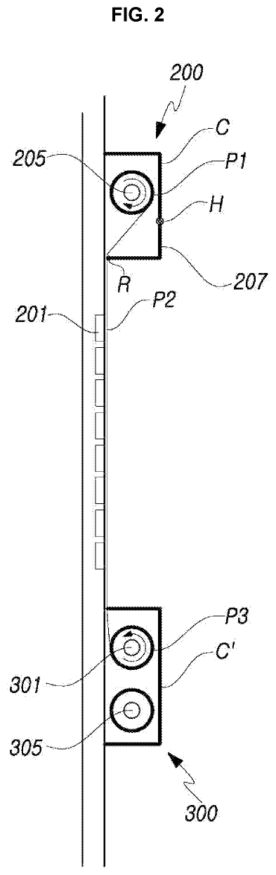

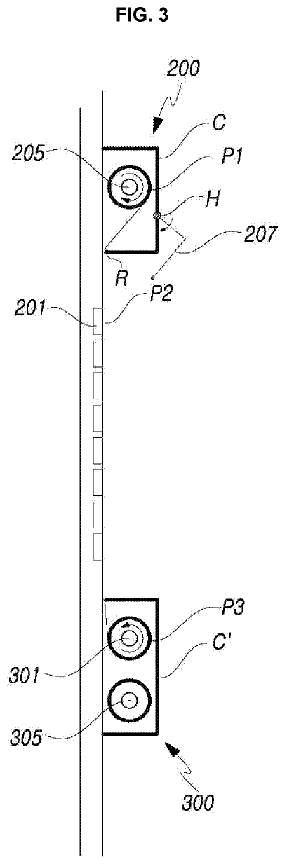

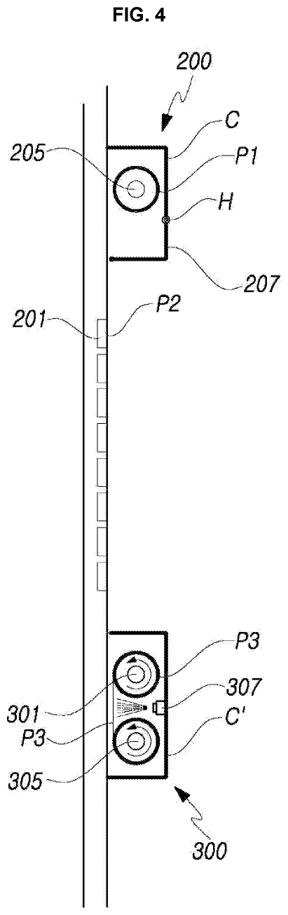

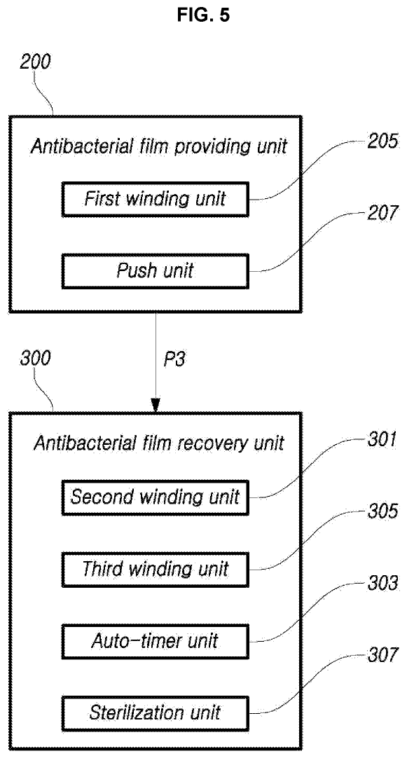

FIG. 1 is a front view showing an example of an apparatus for providing an antibacterial film according to an embodiment of the present disclosure installed on a manipulation panel. FIG. 2 is a side view of FIG. 1. FIG. 3 is a view showing an example of operation of a push unit in FIG. 2. FIG. 4 is a diagram showing a sterilization unit of an antibacterial film recovery unit. FIG. 5 is a block diagram of an apparatus for providing an antibacterial film according to an embodiment of the present disclosure.

As shown in the drawings, an embodiment of the present disclosure provides an apparatus for providing an antibacterial film to a manipulation panel 203 having at least one button 201 used to select a target, including an antibacterial film providing unit 200 including a first winding unit 205 on which an antibacterial film P1 before use is wound, to provide an antibacterial film P2 in use such that the antibacterial film fed from the first winding unit 205 covers the button 201 on the manipulation panel 203; and an antibacterial film recovery unit 300 including a second winding unit 301 on which an antibacterial film P3 after use is wound.

The present disclosure may be applied to automatic beverage vending machines having buttons for choosing ‘desired beverages’ and elevators having internal manipulation panels with buttons for choosing ‘destination floors’, and hereinafter, the present disclosure will be described based on an example of use in a manipulation panel inside an elevator.

The antibacterial film providing unit 200 has the first winding unit 205 on which the antibacterial film P1 before use is wound.

The antibacterial film providing unit 200 may be provided, for example, in the form of a case C in which the first winding unit 205 is received.

The antibacterial film providing unit 200 may be attached to the wall above the manipulation panel 203 by an adhesive tape.

The first winding unit 205 may be provided as, for example, a winding roller having a rotation axis on two sides.

The antibacterial film P1 before use is received in the antibacterial film providing unit 200 in a state that it is wound on the first winding unit 205.

The antibacterial film providing unit 200 provides the antibacterial film P2 in use such that the antibacterial film fed from the first winding unit 205 covers the button 201 on the manipulation panel 203.

Here, the antibacterial film P2 in use refers to an antibacterial film disposed on the button 201 to cover the button 201 to allow a user (a passenger) to contact with his/her finger when the user rides in the elevator and presses the button.

Meanwhile, the antibacterial film providing unit 200 may further include a push unit 207 to press down the upper surface of the antibacterial film at an area of the antibacterial film fed from the first winding unit 205 to bring the antibacterial film fed from the first winding unit 205 into close contact with the button 201 on the manipulation panel 203.

Describing an example of the configuration of the push unit 207, the push unit 207 may have an end that is coupled to the end of the case C pivotably around a hinge axis H and an opposite side that is extended and bent toward the manipulation panel 203, and a press roller R may be coupled to the end.

Although not shown in the drawings, the push unit 207 may further include a locking member to fix the position of the push unit 207 placed as shown in FIG. 2 after the push unit 207 is pivoted around the hinge axis H, and may include an actuator, for example, an electric motor, to automatically pivot the push unit 207 around the hinge axis H by the user's manipulation.

Through the above-described configuration of the push unit 207, the antibacterial film P2 in use disposed on the button 201 to cover the button 201 comes into close contact with the upper surface of the button 201.

The antibacterial film recovery unit 300 has the second winding unit 301 on which the antibacterial film P3 after use is wound.

The second winding unit 301 may be provided as, for example, a winding roller having a rotation axis on two sides.

The antibacterial film P3 after use is received in the antibacterial film recovery unit 300 in a state that it is wound on the second winding unit 301.

The antibacterial film recovery unit 300 may be provided, for example, in the form of a case C′ in which the second winding unit 301 is received.

The antibacterial film recovery unit 300 may be attached to the wall below the manipulation panel 203 by an adhesive tape.

Meanwhile, the antibacterial film recovery unit 300 may further include an auto-timer unit 303 to operate the second winding unit 301 to wind the antibacterial film P3 after use at a set predetermined time interval.

The auto-timer unit 303 may operate the second winding unit 301 to put it into rotation, for example, at a set 24-hour interval, to pull the antibacterial film P2 in use so that the antibacterial film P3 after use is wound on the second winding unit 301.

Through this, a new antibacterial film is disposed on the upper surface of the button 201 at the set predetermined time interval.

Meanwhile, the antibacterial film recovery unit 300 may further include a third winding unit 305 to wind the antibacterial film P3 after use fed from the second winding unit 301; and a sterilization unit 307 to sterilize the antibacterial film P3 after use wound on the third winding unit 305.

The third winding unit 305 may be provided as, for example, a winding roller having a rotation axis on two sides.

The sterilization unit 307 sterilizes the antibacterial film P3 after use wound on the third winding unit 305.

For example, the sterilization unit 307 may include an ultraviolet (UV) light source to emit UV radiation to the antibacterial film P3 after use.

The sterilization unit 307 may be connected to or detachably attached to the inner peripheral surface between the second winding unit 301 and the third winding unit 305 on the inner peripheral surface of the case C′.

Through this, the antibacterial film P3 after use may become a reusable antibacterial film.

As described above, according to an embodiment of the present disclosure, it is possible to place the antibacterial film over the button on the manipulation panel through the antibacterial film providing unit and the antibacterial film recovery unit, thereby preventing the spread of bacteria that may be transmitted by many users' button manipulation.

Additionally, it is possible to automatically replace the antibacterial film that covers the button on the manipulation panel at the predetermined time interval set through the auto-timer unit, thereby improving the convenience of use.

Additionally, the antibacterial film after use may be sterilized through the sterilization unit provided in the antibacterial film recovery unit for reuse, thereby achieving resource savings.

While all the elements that constitute an embodiment of the present disclosure have been hereinabove described as being combined into one or working in combination, the present disclosure is not necessarily limited to such an embodiment. That is, all the elements may work in at least one selective combination within the intended scope of the present disclosure.

The foregoing description is made to describe the technical spirit of the present disclosure by way of illustration, and it is obvious to those skilled in the art that a variety of modifications and changes will be made without departing from the essential features of the present disclosure. Accordingly, the embodiments disclosed herein are provided to describe, but not intended to limit, the technical spirit of the present disclosure, and the technical spirit and scope of the present disclosure is not limited by these embodiments. The scope of protection of the present disclosure should be interpreted by the appended claims, and it should be interpreted that the full technical spirit in the equivalent scope falls within the scope of protection of the present disclosure.

What is claimed is:

1. An apparatus for providing an antibacterial film to a manipulation panel having at least one button used to select a target, the apparatus comprising:
an antibacterial film providing unit including a first winding unit on which an antibacterial film before use is wound, to provide an antibacterial film in use such that the antibacterial film fed from the first winding unit covers the button on the manipulation panel; and
an antibacterial film recovery unit including a second winding unit on which an antibacterial film after use is wound.

2. The apparatus for providing an antibacterial film according to claim 1, wherein the antibacterial film providing unit further includes:
a push unit to press down an upper surface of the antibacterial film at an area of the antibacterial film fed from the first winding unit to bring the antibacterial film fed from the first winding unit into close contact with the button on the manipulation panel.

3. The apparatus for providing an antibacterial film according to claim 1, wherein the antibacterial film recovery unit further includes an auto-timer unit to operate the second winding unit to wind the antibacterial film after use at a set predetermined time interval.

4. The apparatus for providing an antibacterial film according to claim 1, wherein the antibacterial film recovery unit further includes:
a third winding unit to wind the antibacterial film after use fed from the second winding unit; and
a sterilization unit to sterilize the antibacterial film after use wound on the third winding unit.

5. The apparatus for providing an antibacterial film according to claim 4, wherein the sterilization unit includes an ultraviolet light source to emit ultraviolet radiation to the antibacterial film after use.
